(19) United States
(12) Patent Application Publication
(10) Pub. No.: US 2025/0017487 A1
(43) Pub. Date: Jan. 16, 2025

(54) RESPIRATION SENSOR SYSTEM WITH NANOCOMPOSITE STRUCTURE

(71) Applicant: HACETTEPE UNIVERSITESI REKTORLUK, Cankaya/Ankara (TR)

(72) Inventors: Dincer GOKCEN, Ankara (TR); Yeter SEKERTEKIN, Ankara (TR)

(73) Assignee: HACETTEPE UNIVERSITESI REKTORLUK, Cankaya/Ankara (TR)

(21) Appl. No.: 18/712,821

(22) PCT Filed: Nov. 23, 2022
(86) PCT No.: PCT/TR2022/051343

§ 371 Date: May 23, 2024

(30) Foreign Application Priority Data
Nov. 23, 2021 (TR) 2021/018260

(51) Int. Cl. A61B 5/08

(52) U.S. Cl. CPC A61B 5/0816; A61B 2562/125

(57) ABSTRACT

The present invention relates to a system for tracking the respiration of a user who wears a mask in which a nanocomposite based electrode, that has a serpentine geometry, is screen-printed on a flexible filter substrate, and the resistance of which varies with the temperature and humidity of the air passing through the substrate, is inserted.

### TECHNICAL FIELD

The present invention relates to a system for tracking the respiration of a user who wears a mask in which a nanocomposite-based electrode, that has a serpentine geometry, is screen-printed on a flexible filter substrate, and the resistance of which varies with the temperature and humidity of the air passing through the substrate, is inserted.

### BACKGROUND OF THE INVENTION

Respiration sensors are applied as a simple pressure sensor which is inserted around rib cage by means of a belt and measures the change of amplitude in user's cycles of inhalation-exhalation. It is possible to use the related sensor in patients who have deep breathing ability or do not suffer from acute or chronic shortness of breath and it cannot be used to track the said patients suffering from short of breath due to sensor characteristics.

From patients suffering from respiratory difficulties due to simple respiratory tract infections to patients suffering from respiratory difficulties due to chronic lung diseases in daily life, high relative humidity is observed in exhaled breath compositions and the relative humidity decreases in inhaled breath during inhalation-exhalation of all users. Tracking a user's inhalation-exhalation function can be ensured with stable measurement of relative humidity.

Considering the deficiencies of respiration sensors included in the current technique, it is understood that there is need for a system for tracking the respiration of a user who wears a mask in which a nanocomposite-based electrode, that has a serpentine geometry, is screen-printed on a flexible filter substrate, and the resistance of which varies with the temperature and humidity of the air passing through the substrate, is inserted to keep track of the respiration of a user who wears a mask in which a nanocomposite-based electrode, that has a serpentine geometry, is screen-printed on a flexible filter substrate, and the resistance of which varies with the temperature and humidity of the air passing through the substrate, is inserted.

The article document titled “Paper-Based Respiration Sensor” and included in the state of the art discloses a paper-based respiration sensor system. The said system comprises a flexible electrode couple and a resistance sensor with extensions which are printed on pure cellulose paper by means of two-dimensional printing techniques and are embedded; the pure cellulose-based electrode, that is inserted into the inhalation-exhalation area, measures the resistance value which increases and decreases according to the change of humidity around the cellulose fibers; and enables to track the cycle of inhalation-exhalation which is matched with the resistance value

### SUMMARY OF THE INVENTION

The objective of the present invention is to realize a system for tracking the respiration of a user who wears a mask in which a nanocomposite-based electrode, that has a serpentine geometry, is screen-printed on a flexible filter substrate, and the resistance of which varies with the temperature and humidity of the air passing through the substrate, is inserted.

### DETAILED DESCRIPTION OF THE INVENTION

“Respiration Sensor System with Nanocomposite Structure” realized to fulfill the objectives of the present invention is shown in the FIGURES attached, in which:

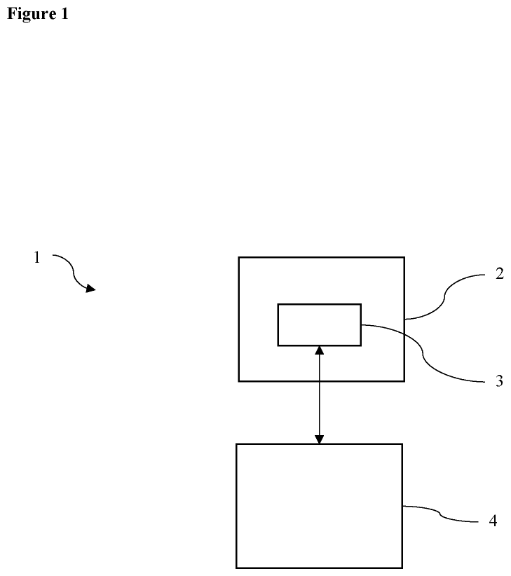

FIG. 1 is a schematic block diagram of the inventive device.

The components illustrated in the FIGURE are individually numbered, where the numbers refer to the following:
1. System
2. Flexible filter substrate
3. Nanocomposite serpentine electrode
4. Resistance meter

The inventive system (1) for tracking the respiration of a user who wears a mask in which a nanocomposite-based electrode, that has a serpentine geometry, is screen-printed on a flexible filter substrate, and the resistance of which varies with the temperature and humidity of the air passing through the substrate, is inserted comprises to keep track of the respiration of a user who wears a mask in which a nanocomposite-based electrode, that has a serpentine geometry, is screen-printed on a flexible filter substrate, and the resistance of which varies with the temperature and humidity of the air passing through the substrate, is inserted comprises:
at least one flexible filter substrate (2) which is configured to be produced from a flexible material with a pore width suitable for filtering purpose, and to be sized so as to be used within a mask;
at least one nanocomposite serpentine electrode (3) which is configured to be created on the flexible filter substrate (2); and
at least one resistance meter (4) which is configured to connect to at least one nanocomposite serpentine electrode (3) electronically, to measure and then report the resistance values of electrodes.

The flexible filter substrate (2) included in the inventive system (1) is configured to be produced from a flexible material with a pore width suitable for filtering purpose, and to be sized so as to be used within a mask. In a preferred embodiment of the invention, the flexible filter substrate (2) is configured to be produced as an interlining-based mask material combined with meltblown technique.

The nanocomposite serpentine electrode (3) included in the inventive system (1) is configured to be created on the flexible filter substrate (2). In a preferred embodiment of the invention, the nanocomposite serpentine electrode (3) is configured to be printed in a thin and flexible structure with a serpentine geometry by means of screen printing processes as nanocomposite-based. In a preferred embodiment of the invention, the nanocomposite serpentine electrode (3) is configured to connect to a measurement unit electronically.

The resistance meter (4) included in the inventive system (1) is configured to connect to at least one nanocomposite serpentine electrode (3) electronically, to measure and then report the resistance values of electrodes. In a preferred embodiment of the invention, the resistance meter (4) is configured to save the resistance value of the nanocomposite serpentine electrode (3) that varies by the value of air temperature and relative humidity passing through the flexible filter substrate (2).

### INDUSTRIAL APPLICABILITY OF THE INVENTION

In the inventive system (1), at least one nanocomposite serpentine electrode (3)—which is printed on the flexible filter substrate (2), that is produced to be turned into a protective mask, in a thin and flexible structure with a serpentine geometry by means of screen printing processes as nanocomposite-based—is connected to the resistance meter (4) electronically; the resistance value of at least one nanocomposite serpentine electrode (3)—it's temperature and relative humidity passing through the flexible filter substrate (2) vary by the air that is different from the environment every time the user inhales and exhales—is saved by the resistance meter (4). The saved data can be analyzed and the user's inhalation-exhalation information can be interpreted.

With the inventive system (1), it is possible to save a user's inhalation-exhalation pattern by means of the nanocomposite serpentine electrode (3) which is adapted on a protective mask that can be used by all users and the resistance of which is changed by the changing temperature and the relative humidity during inhalation-exhalation.

Within these basic concepts; it is possible to develop a wide variety of embodiments of the inventive “Respiration Sensor System (1) with Nanocomposite Structure”; the invention cannot be limited to examples disclosed herein and it is essentially according to claims.

1. A system (1) for tracking the respiration of a user who wears a mask in which a nanocomposite-based electrode, that has a serpentine geometry, is screen-printed on a flexible filter substrate, and the resistance of which varies with the temperature and humidity of the air passing through the substrate, is inserted; comprising
at least one flexible filter substrate (2) which is configured to be produced from a flexible material with a pore width suitable for filtering purpose, and to be sized so as to be used within a mask; and characterized by
at least one nanocomposite serpentine electrode (3) which is configured to be created on the flexible filter substrate (2); and
at least one resistance meter (4) which is configured to connect to at least one nanocomposite serpentine electrode (3) electronically, to measure and then report the resistance values of electrodes.

2. A system (1) according to claim 1; characterized by the flexible filter substrate (2) which is configured to be produced as an interlining-based mask material combined with meltblown technique.

3. A system (1) according to claim 1; characterized by the nanocomposite serpentine electrode (3) which is configured to be printed in a thin and flexible structure with a serpentine geometry by means of screen printing process of nanocomposite.

4. A system (1) according to claim 1; characterized by the nanocomposite serpentine electrode (3) which is configured to connect to a measurement unit electronically.

5. A system (1) according to claim 1; characterized by the resistance meter (4) which is configured to save the resistance value of the nanocomposite serpentine electrode (3) that varies by the value of air temperature and relative humidity passing through the flexible filter substrate (2).
